(19) United States
(12) Patent Application Publication
(10) Pub. No.: US 2007/0202056 A1
(43) Pub. Date: Aug. 30, 2007

(54) Oral composition for improving mouth environment

(76) Inventors: Paul Losee, Layton, UT (US); Richard Austin, Layton, UT (US); Blaine D. Austin, Layton, UT (US)

Correspondence Address: JAMES SONNTAG;JAMES SONNTAG, PATENT ATTORNEY, P.O. BOX 9194, SALT LAKE CITY, UT, 84109, US

(21) Appl. No.: 11/377,223

(22) Filed: Mar. 14, 2006

Related U.S. Application Data
(60) Provisional application No. 60/661,411, filed on Mar. 14, 2005.

(51) Int. Cl. A61K 9/68; A61K 8/22

(52) U.S. Cl. 424/48; 424/53

(57) ABSTRACT

A solid oral composition for providing a mouthwash efficacious effect comprising a hygroscopic base, carbamide peroxide, and a bacterial immobilizing agent.

### CROSS REFERENCE TO RELATED APPLICATIONS

This application claims priority from U.S. Provisional Application 60/661411, filed 14 Mar. 05, which is hereby incorporated by reference.

### BACKGROUND OF INVENTION

Compositions for whitening teeth are have been made. Examples of such compositions are disclosed in U.S. Pat. No. 6,149,211, Ser. No. 09/028330, issued 21 Nov. 2000, to Losee et al., which is hereby incorporated by reference.

### SUMMARY OF INVENTION

The present invention involves a composition and method for manufacture for a solid orally administered formulation. The formulation is water-free and the ingredients are carbamide peroxide, a bacterial immobilizing agent, and a base of a non-hygroscopic sweetener, which may be mannitol, or a hydrogenation product of isomaltulose, such as the disaccharide, Isomalt™ or mixtures thereof. The formulation is stable, having a shelf life of several weeks.

The composition of the invention has the effect of improving the mouth environment by, among other things, raising the pH and reducing bacterial growth (which reduces cavity formation and gingivitis, and improves the healing of wounds). The compositions of present invention are useful in the prevention and reduction of bad breath, plaque and related gum diseases, and effective in killing, and/or altering the bacterial metabolism, and/or for a period of time suppressing the growth to microorganisms which cause topically-treatable infections and diseases of the oral cavity, such as plaque, gingivitis, periodontal disease, and breath malodor.

The present invention provides the same effect as a liquid mouthwash, but with a solid composition taken orally in the form of a tablet or the like.

Without being bound to any theory, it is believed that the combination of the non-hygroscopic sweetener, the carbamide peroxide and the bacterial immobilizing agent provide an enhanced efficacy and act together to synergistically improve the mouth environment.

It have been found that compositions of a non-hygroscopic sweetener base, and carbamide peroxide (as disclosed in U.S. Pat. No. 6,149,211) provide some improvement to the mouth environment, but it is believed that these components in combination with a suitable bacterial immobilizing agent or agents provide an unexpected improvement.

The composition of the invention does not degrade and has a long shelf-life. But, as the product is orally administered and is dissolved by the saliva while being chewed or held in the mouth, it reacts with the saliva to produce an effect that improves the mouth environment without producing an unpleasant taste. It has been found that any urea that is formed in this process of dissolution in the mouth is insufficient to impart a bad taste to the product.

### BRIEF DESCRIPTION OF THE DRAWINGS

FIG. 1 is a flow-sheet of a method of the invention.

FIG. 2 is a flow-sheet of a method of the invention.

### DETAILED DESCRIPTION OF THE INVENTION

The compositions of the invention comprise the ingredients, urea carbamide, a bacterial immobilizing agent and a non-hygroscopic base or sweetener composition.

Non-Hygroscopic Sweetener Base

For purposes of this disclosure the base composition is the portion of the composition of the invention other than the carbamide peroxide and the bacterial immobilization agent. The major portion (>50 wt. %) of base composition comprises a non-hygroscopic sweetener base as the sole or principal sweetener. This may be one of or a mixture of mannitol, and a hydrogenation product of isomaltulose. The base also comprises other materials used conventionally to form the appropriate paste, gum, tablet or candy. The base is essentially water-free and non-hygroscopic. By “water-free” is meant that it contains negligible water that is free to react with the carbamide peroxide. Components of the composition may contain waters of hydration, but they must be bound sufficiently to the base composition or be present in such a small amount to provide negligible reactive water.

By “non-hygroscopic” is meant a composition lacking a hygroscopic property, i.e., the tendency of the substance to hydrate by absorbing water, usually from the ambient humidity. The base material should be non-hygroscopic at ambient temperatures and humidities usually encountered during handling and storage. The composition of the invention may comprise materials that are hygroscopic, but they must be present in small enough amounts, such that the essentially non-hygroscopic nature of the entire base composition is retained.

A suitable composition for the sweetener of the base composition is mannitol, a catalytically hydrogenation product of isomaltulose (6-0-.alpha.-D-Glucopyranosyl-D-fructose), or mixtures thereof.

The catalytic hydrogenation of isomaltulose (a derivative of sucrose) produces two stereoisomers, 6-0-.alpha.-D-Glucopyranosyl-D-sorbitol (1,6-GPS) and 1-0-.alpha.-D-Glucopyranosyl-D-mannitol (1,1-GPM). (These are also known as “-D-glucopyranosyl-1,6-D-sorbitol and “-D-glucopyranosyl-1,1-D-mannitol, respectively.) The hydrogenation reaction is an equimolar mixture of the two isomeric disaccharide polyols, 1,6-GPS and 1,1-GPM. Isomalt is sold under the trademark Palatinit.TM. by Palatinit Sussungsmittel GmbH, Mannheim, Germany. Isomalt is non-hygroscopic, particularly at ambient temperatures and humidities. At 25 degrees C., Isomalt absorbs very little water. At temperatures as high as 60 degrees C. and 80 degrees C. the relative humidity must be 75% and 65% respectively for significant water absorption. A detailed discussion of Isomalt, its manufacture and various properties is disclosed in Isomalt, by Peter Strater and William E. Irwin in Alternative Sweeteners, edited by Lyn O'Brien Nabors and Robert C. Gellardi, Marcel Dekker, Inc., 1992, and ISOMALT, 5th edition, published in 1996 by Platinit Sussungsmittel GmbH, Gottlieb-Daimler-Stra.beta.e, D-68165, Mannheim, Germany. The hydrogenation reaction of isomaltulose is disclosed in U.S. Pat. No. 4,117,173 to Schiweck et al.

The base composition may comprise other suitable ingredients that are used in candy and tablet compositions, such as other non-hygroscopic sweeteners, flavors, sweeteners, anti-oxidants, stabilizers, colorants, and the like.

Carbamide Peroxide

An active ingredient in the present invention is carbamide peroxide, CON₂H₄.H₂O₂. It is the addition compound of hydrogen peroxide and urea and is also known as urea peroxide, or urea hydrogen peroxide.

The composition of the invention contains carbamide peroxide in an effective amount, i.e., an amount to be efficacious to improve the mouth environment in combination with the bacterial immobilization agent when the composition is taken orally and dissolved in the mouth or chewed to dissolve and release the carbamide peroxide and bacterial immobilization agent. An efficacious amount includes amounts of carbamide peroxide below about 3 weight percent, and below those amounts below where the carbamide peroxide provides a tooth-whitening effect. An aspect of the invention includes compositions comprising carbamide peroxide and a non-hygroscopic base where the carbamide peroxide is in an amount insufficient to provide an effective or satisfactory tooth-whitening effect, but sufficient to provide an efficacious amount for bacterial immobilization.

Bacterial Immobilizing Agent

The present invention includes a bacterial immobilizing agent. By “bacterial immobilizing” is meant prevention, suppression, or inhibition of bacterial growth. Thus, for the bacterial immobilizing agent bactericidal and bacteriostatic compositions or mixtures thereof are suitable. These include antibacterial agents often commonly employed in oral compositions and that are compatible with the other components in the non-hygroscopic environment of the composition and are activated in the mouth environment. Specific examples include, but are not limited to, chlorine dioxide, and biguanides, such as chlorhexidine digluconate. Examples may also possibly include quaternary ammonium antibacterial compounds and water-soluble sources of certain metal ions such as zinc, copper, silver and stannous (e.g., zinc, copper and stannous chloride, and silver nitrate).

Method of Manufacture

The composition of the invention is made by any suitable process where carbamide peroxide is incorporated into the solid base material such that the carbamide peroxide is protected or shielded from humidity in the atmosphere, and no water or no hygroscopic materials that would absorb water are introduced that would result in water being introduced into the composition during processing or storage in an amount that would produce a taste-detectable amount of urea over several days or weeks.

Carbamide peroxide when reacted with water decomposes into urea. In the compositions of the present invention, the carbamide peroxide is isolated from water while in the product, so that it cannot react with water. This is accomplished by not adding any water during manufacture of the composition, and using materials that are as free from water as practical. In addition, the components of the composition are preferably non-hygroscopic and do not attract water from the atmosphere. In addition, the composition must have a physical and chemical nature that protects the carbamide peroxide from humidity in the air. It has been found, that some oral compositions with certain non-hygroscopic sweeteners are unsatisfactory and bad-tasting. While not completely understood, it is believed that the unsatisfactory taste in these certain products may be due to an unsatisfactory taste of the sweetener itself, and/or from urea produced because the base material does not adequately isolate the carbamide peroxide from atmospheric moisture. Thus, the non-hygroscopic nature of the sweetener-base is the most important, but not the only factor. However, it has been found that when a hydrogenation product of isomaltulose and/or mannitol is used as the sweetener base, the urea carbamide is stabilized and protected and will not degrade over several weeks of storage.

Certain prior-art gum, tablet and candy compositions contain water, or often contain substances that attract water from the humidity in the air. In normal gum, tablet and candy compositions, this water is no problem. But if carbamide peroxide is present, that water is available to react with and degrade the carbamide peroxide to urea, which is bad tasting even in very small concentrations.

In the method of the present invention, materials that are essentially free of water are used, or water is expelled from the composition. Because of the inherent properties of the base of the composition of the invention, there is no readsorbtion or attraction of water from the humidity in the air. Accordingly, the carbamide peroxide is protected or shielded from water and the decomposition reaction does not take place sufficiently to produce an unpleasant taste. Accordingly, it has been found that the compositions of the present invention can be stored for several weeks with no detectable deterioration in taste and with an insignificant loss of the active ingredient.

When the composition of the invention is placed within the mouth and is allowed to dissolve or is chewed, the composition slowly dissolves in the mouth, which provides an effective amount of the carbamide peroxide and bacterial immobilizing agent to improve the environment of the mouth. Preferably, a gum, candy or tablet of the invention is formed so as to dissolve the carbamide peroxide and bacterial immobilizing agent slowly over about a 20 minute period. A consistent daily use of the tablets of the invention will then obtain a useful effect for the consumer.

One method for manufacturing the composition of the invention comprises first heating the base material to a temperature sufficient to drive off any water in the composition. For Isomalt, this is a temperature of about 150 degrees C. or above. The base material is then cooled to a temperature at which the carbamide peroxide and the bacterial immobilizing agent can be incorporated and mixed into the base material. As the temperature approaches near is 120 degrees C., the carbamide peroxide disassociates. Accordingly, the preferred mixing temperature is 118 degrees C. or below. When Isomalt cools to near 110 degrees C. it becomes difficult to mix the carbamide peroxide in an Isomalt base. Accordingly the preferred temperature for mixing in Isomalt is 114 degrees C. or above. In summary, for mixing carbamide peroxide in Isomalt, the mixing temperature is preferably between 114 degrees Celsius and 118 degrees C., more preferably about 116 degrees C.

After incorporating the carbamide peroxide and the bacterial immobilizing agent, the material is then allowed to solidify. The solid base can be formed into hard candy shapes by any suitable method, or crushed into powder and then hard pressed in to tablets, or otherwise processed by conventional methods into a solid form that can be orally administered. Basically, any process that permits the heating to expel the water and the mixing temperature of the carbamide peroxide is suitable for the present invention. The other components of the composition, i.e., the sweeteners, stabilizers, flavors, etc. are added at any suitable time, as dictated by their properties.

Alternately, a composition of the invention may be produced by compressing into the tablets a powdered base material (as defined above), a bacterial immobilization agent, and a powdered carbamide peroxide.

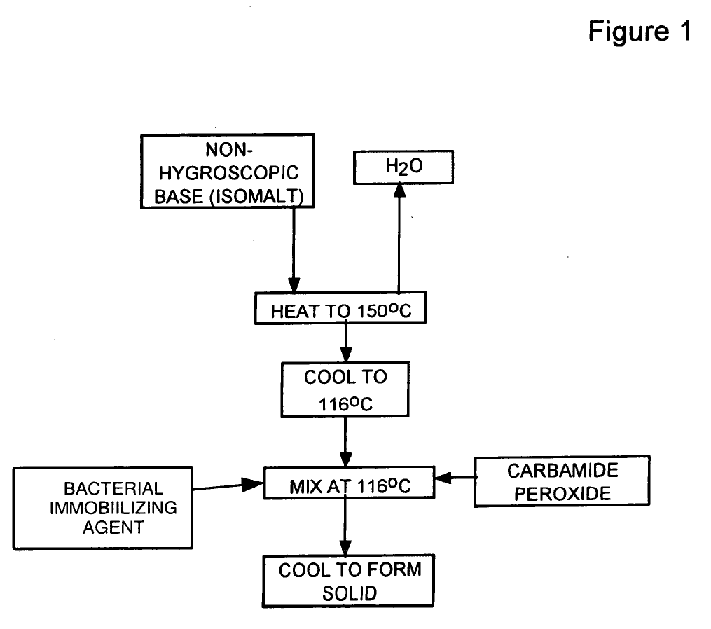

Referring to FIG. 1, which is a flow sheet of a method for manufacture of the composition of the invention, a non-hygroscopic base, as defined herein, is provided and heated to 150 degrees C. to drive of the water in the base. The base is cooled to 116 degrees C., the temperature at which carbamide peroxide is added and mixed. The material is then allowed to cool to form a solid material.

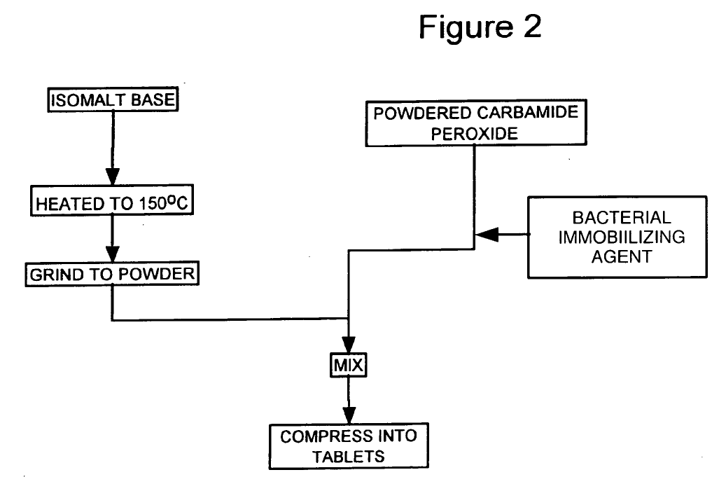

FIG. 2 is a flow sheet of an alternate method for manufacture of the composition of the invention. A non-hygroscopic base is provided optionally heated to 150 degrees C. to drive off moisture. The base is then ground and mixed with powdered carbamide peroxide and the mixture is compressed into tablets by conventional technique

While this invention has been described with reference to certain specific embodiments and examples, it will be recognized by those skilled in the art that many variations are possible without departing from the scope and spirit of this invention, and that the invention, as described by the claims, is intended to cover all changes and modifications of the invention which do not depart from the spirit of the invention.

1. A non-hygroscopic solid oral composition comprising:
carbamide peroxide in an amount effective to dissolve and produce a efficacious improvement in mouth environment when the composition is dissolved in mouth;
bacterial immobilizing agent;
a solid, water-free, non-hygroscopic base;
with water in the composition at sufficiently low amount and the composition being sufficiently non-hygroscopic to avoid a detectable taste of urea from the reaction of carbamide peroxide with water.

2. A non-hygroscopic solid oral composition as in claim 1 wherein the bacterial immobilizing agent is one of or a mixture of chlorine dioxide, or chlorhexidine digluconate.

3. A non-hygroscopic solid oral composition as in claim 1 wherein the non-hygroscopic base comprises a hydrogenation product of isomaltulose.

4. A non-hygroscopic solid oral composition as in claim 1 wherein the non-hygroscopic base comprises mannitol.

5. A non-hygroscopic solid oral composition as in claim 1 wherein the composition is in the form of a tablet.

6. A non-hygroscopic solid oral composition as in claim 1 wherein the composition is in the form of a candy.

7. A non-hygroscopic solid oral composition as in claim 1 wherein the composition is in the form of a gum.

8. A non-hygroscopic solid oral composition comprising:
carbamide peroxide in an amount effective to dissolve and produce a efficacious improvement in mouth environment when the composition is dissolved in mouth and in an amount insufficient to provide a tooth whitening effect;
a solid, water-free, non-hygroscopic base;
with water in the composition at sufficiently low amount and the composition being sufficiently non-hygroscopic to avoid a detectable taste of urea from the reaction of carbamide peroxide with water.

9. A non-hygroscopic solid oral composition as in claim 8 additionally comprising a bacterial immobilizing agent that with the carbamde peroxide provides an efficacious improvement in mouth environment.

10. A non-hygroscopic solid oral composition as in claim 9 wherein the bacterial immobilizing agent is one of or a mixture of chlorine dioxide, or chlorhexidine digluconate.

11. A method for manufacturing a solid oral composition comprising compounding into a mixture an efficacious amount of carbamide peroxide, a bacterial immobilizing agent, and a non-hygroscopic base in a water-free environment, forming the mixture into a solid shape.

12. A method for manufacturing a solid oral composition as in claim 11 wherein the water-free, non-hygroscopic base comprises one of or a mixture of mannitol, and a hydrogenation product of isomaltulose, with water in the composition at sufficiently low amount and the composition being sufficiently non-hygroscopic to avoid a detectable taste of urea from the reaction of carbamide peroxide with water, the method for manufacturing additionally comprising forming the base and carbamide peroxide into a solid shape.
